(19) United States
(12) Patent Application Publication
(10) Pub. No.: US 2025/0186654 A1
(43) Pub. Date: Jun. 12, 2025

(54) CROSSLINKED STRUCTURAL ORTHOPEDIC BIOMATERIAL AND METHOD FOR MANUFACTURE

(71) Applicant: Orthomod LLC, Dayton, OH (US)

(72) Inventors: Shawn A. Hunter, Springboro, OH (US); David Louis Kirschman, Dayton, OH (US); Dewey J. Weeda, Beavercreek, OH (US)

(21) Appl. No.: 18/974,495

(22) Filed: Dec. 9, 2024

Related U.S. Application Data
(60) Provisional application No. 63/607,768, filed on Dec. 8, 2023.

(51) Int. Cl. A61L 27/44; A61L 27/46

(52) U.S. Cl. CPC A61L 27/446; A61L 27/46; A61L 2300/112; A61L 2430/02; A61L 2430/12

(57) ABSTRACT

Provided herein are surgical implants and methods of manufacturing the surgical implants. Generally, the method includes polymerizing a bifunctional monomer with a long-chain acrylic to form a high-strength copolymer, dispersing a plurality of ceramic particles in the copolymer to form a composite biomaterial, forming the composite biomaterial into an implant, and crosslinking the formed implant to form the surgical implant, thereby stabilizing the ceramic particles in the surgical implant.

### CROSS-REFERENCES TO RELATED APPLICATIONS

This application claims the benefit of priority to U.S. Provisional Application No. 63/607,768, filed Dec. 8, 2023.

### TECHNICAL FIELD

The present disclosure relates generally to the field of surgical implants and methods of manufacturing the surgical implants, more specifically, to high-strength polymer implants.

### BACKGROUND

Orthopedic implants are used for replacing or augmenting damaged or diseased bone. Conventional implants made from metals, ceramics, or polymers lack the optimal combination of manufacturability, mechanical strength, and osteointegration. Particularly, the development of polymeric implants that balance these characteristics remains a significant challenge, particularly when resistance to long term cyclical loading is required, such as in spinal implants or hip prostheses.

Accordingly, a need exists for surgical implants and manufacturing methods that effectively balance mechanical strength, osteointegration, and manufacturability.

### SUMMARY

This present disclosure relates to surgical implants and methods of manufacturing the implants.

In one embodiment, the present disclosure relates to a method of manufacturing a surgical implant, optionally an orthopedic or dental implant, the method comprising polymerizing a bifunctional monomer with a long-chain acrylic to form a high-strength copolymer; dispersing a plurality of ceramic particles in the copolymer to form a composite biomaterial; forming the composite biomaterial into an implant; and crosslinking the formed implant to form the surgical implant, thereby stabilizing the ceramic particles in the surgical implant. In some embodiments, the bifunctional monomer is selected from allyl methacrylate, vinyl acetate, ethylene glycol dimethacrylate, 1,4-butanediol dimethacrylate, glycidal methacrylate, divinylbenzene, and combinations thereof, optionally allyl methacrylate. In some embodiments, the long-chain acrylic is selected from polymethyl methacrylate, polymethyl methacrylate, polybutyl methacrylate, polylaurel methacrylate, polyethylene methacrylate, polystyrene methacrylate, polymethyl acrylate, and combinations thereof, optionally, polymethyl methacrylate. The high-strength polymer may have a molecular weight of about 100,000 Daltons. Optionally, the ceramic particles are selected from hydroxyapatite, tricalcium phosphate, bioglass, silicates and/or zirconia. In some embodiments, the plurality of ceramic particles are dispersed in the copolymer when the copolymer is in a molten state.

In some embodiments, wherein crosslinking the formed implant comprises diallyl crosslinking. Optionally, crosslinking is initiated by applying heat, pressure, irradiation (such as gamma irradiation, ultraviolet irradiation, microwave radiation, electron beam irradiation, infrared radiation, and combinations thereof), or a combination thereof to the formed implant. In some embodiments, the high-strength copolymer and/or the composite biomaterial is formed with a catalyst, optionally peroxides, UV initiators, or metal-based catalysts, to facilitate crosslinking.

In some embodiments, crosslinking the formed implant enhances one or more properties of the surgical implant relative to the formed implant, optionally selected from mechanical properties, such as increased tensile strength or increased compressive strength, thermal properties, or chemical properties.

### DETAILED DESCRIPTION

The above described deficiencies are addressed by the present disclosure, which combines post-polymerization crosslinking of an implant formed with a high-strength polymer for use as a surgical or dental implant in a subject.

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used in the specification and the appended claims, the singular forms “a,” “an,” and “the” are intended to include the plural forms as well, unless the context clearly indicates otherwise. Thus, for example, reference to “a” component includes aspects having two or more such components, unless the context clearly indicates otherwise.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Embodiments of the present disclosure are directed to orthopedic biomaterials. In some embodiments, the orthopedic biomaterials may be adapted for use as medical or dental implants. Optionally, the orthopedic biomaterials include a high-strength copolymer. The copolymer's properties, described in greater detail herein, allow for the use of standard manufacturing techniques, including molding, extrusion, and 3D printing, facilitating the creation of implants with complex and patient-specific geometries. In some embodiments, the high-strength copolymer is formed by polymerizing a monomer with a long-chain acrylic to form a high-strength copolymer.

In some embodiments, the high-strength copolymer is an acrylic copolymer. The high-strength copolymer may include a monomer with a plurality of functional groups capable of participating in polymerization or other chemical reactions, such as 2 functional groups, 3 functional groups 4 functional groups, etc. It will be appreciated that a monomer with a plurality of functional groups may polymerize to form a polymer matrix using one functional group, while leaving one or more functional groups available for post-polymerization crosslinking, described in greater detail herein. The functional groups may be identical or different. It will be appreciated that the functional groups determine the polymerization patterns of the monomer and may be chosen based on the polymerization conditions (e.g., free-radical, condensation, etc.) Any suitable monomer is contemplated and possible.

In some embodiments, the high-strength copolymer includes a bifunctional monomer. In some embodiments, the high-strength copolymer is formed by polymerizing the bifunctional monomer with a long-chain acrylic to form the high-strength copolymer. As used herein “bifunctional monomer” and grammatical equivalents thereof refers to a monomer having two reactive functional groups capable of participating in polymerization or other chemical reactions. Illustrative, non-limiting examples of suitable bifunctional monomers include allyl methacrylate, vinyl acetate, ethylene glycol dimethacrylate, 1,4-butanediol dimethacrylate, glycidal methacrylate, divinylbenzene, combinations thereof and the like. Optionally, the bifunctional monomer is allyl methacrylate.

In some embodiments, the high-strength copolymer includes a long-chain acrylic. Illustrative, non-limiting examples of suitable long-chain acrylics include polymethyl methacrylate, polyethyl methacrylate, polybutyl methacrylate, polylaurel methacrylate, polyethylene methacrylate, polystyrene methacrylate, polymethyl acrylate, and the like. Optionally, the long-chain acrylic is polymethyl methacrylate.

Optionally, the high-strength copolymer includes a bifunctional monomer and a long chain acrylic. For example, and without being bound by theory, methyl methacrylate and allyl methacrylate may be polymerized to form a copolymer of allyl methacrylate and polymethyl methacrylate. Optionally, during polymerization, the methacrylate groups of both the allyl methacrylate and polymethyl methacrylate polymerize to form the high-strength copolymer, while leaving the allyl groups substantially unreacted. In some embodiments, the unreacted allyl groups function to cross-link the polymer after polymerization, described in greater detail herein.

Any suitable polymerization reaction and/or technique may be used to form the composite matrix. Illustrative, non-limiting examples of polymerization reactions include, but are not limited to addition polymerization (free radical polymerization, cationic polymerization, anionic polymerization, coordination polymerization, etc.), condensation polymerization, ring-opening polymerization, living polymerization, photopolymerization, electrochemical polymerization, combinations thereof, and the like. Various polymerization techniques may be employed, including solution polymerization, bulk polymerization with controlled crosslinking, suspension polymerization, and/or emulsion polymerization. In some embodiments, the polymerization technique is solution polymerization.

Optionally, addition polymerization is used to form the high-strength copolymer. Polymerization generally includes an initiation of the process. It will be appreciated that, in free radical polymerization, initiators, such as azobisisobutyronitrile, benzoyl peroxide, cumene hydroperoxide, and the like, decompose under heat or light to produce free radicals. For example, and without being bound by theory, in some embodiments, azobisisobutyronitrile may thermally decompose to yield two free radicals, which then react with the methacrylate functional groups, initiating the polymer chain. In cationic polymerization, initiators such as Lewis acids or protonic acids generate cations that react with the functional groups which contain electron-rich double bonds. In anionic polymerization, initiators like alkali metals or strong bases produce anions that react with functional groups featuring electrophilic sites. It will be appreciated that initiation may be influenced by various factors, including temperature, which may affect the rate of initiator decomposition and radical generation, the concentration of the initiator, which may impact the number of radicals produced, and/or light exposure for photoinitiators.

It will be appreciated that the selection of a specific initiator may be to influence the rate of polymerization, the polymerization temperature, the molecular weight distribution of the polymer, and/or the overall properties of the final product. It will be further appreciated that the choice of initiator and its decomposition rate may influence the polymer's microstructure, including branching, cross-linking, and stereochemistry. As described in greater detail herein, the initiator may use a different mechanism to initiate polymerization than is used to crosslink the formed implant.

Once the polymerization is initiated, the active site on the growing polymer chain, often a free radical, cation, or anion, reacts with the double bond of a monomer molecule. This reaction results in the formation of a new covalent bond between the monomer and the polymer chain, thereby extending the chain length. During chain propagation, the active site on the growing polymer chain remains reactive, allowing it to continue reacting with additional monomer molecules. The efficiency of propagation may be affected by the stability of the radicals and the availability of monomers. Similarly, in cationic and anionic polymerizations, the active center is a cation or anion that facilitates the addition of monomers to the growing chain through nucleophilic or electrophilic mechanisms. In some embodiments, chain propagation continues until the monomer supply is exhausted or until the polymer chain is terminated. It will be appreciated that controlling factors such as monomer concentration, reaction temperature, and initiator concentration, impacts chain propagation, thereby determining the molecular weight and overall properties of the polymer.

In some embodiments, producing the high-strength copolymer includes controlling the temperature and/or viscosity of the reaction mixture to allow solution polymerization. The monomer and/or the long-chain acrylic may be dissolved in a solvent along with an initiator to form a reaction mixture. Any suitable solvent may be used, including, but not limited to ethanol, methanol, isopropanol, acetone, dimethyl sulfoxide, ethyl acetate, and/or acetonitrile. It will be appreciated that the choice of solvent may impact the dissolution of the monomer, long-chain acrylic, and/or initiator, facilitate homogenization before polymerization, and/or provide a medium for reaction and/or polymerization. For example and without being bound by theory, the solvent may act as a vehicle to dissolve the monomer and initiator, ensuring a uniform reaction mixture. It will be appreciated that a uniform reaction mixture may lead to a more consistent polymer.

In some embodiments, the reaction mixture is maintained at a specific temperature or within a range of temperatures. In some embodiments, the reaction mixture is maintained with a specific viscosity or within a range of viscosity to facilitate polymerization in the solution. In some embodiments, the reaction mixture is maintained at a specific temperature or within a range of temperatures and with a specific viscosity or within a range of viscosity to facilitate polymerization in the solution.

It will be appreciated that the temperature of the reaction mixture may impact the polymerization process and the end characteristics of the polymer. For example, and without being bound by theory, at higher temperatures, the increased rate of initiation and propagation may lead to more polymer chains being formed. However, the increased termination rates can reduce the molecular weight by terminating chains more quickly. Further, the temperature may also affect the structural properties of the resulting polymer. For instance, at higher temperatures, the increased mobility of the growing polymer chains may lead to different microstructures, such as tacticity (the arrangement of substituent groups along the polymer chain) and branching.

Temperatures and/or temperature ranges of polymerization reactions generally allow the reaction to proceed without thermal degradation and/or side reactions. In some embodiments, the temperature range is between 50° C.-100° C. However, the temperature range may be adjusted based on the type of polymerization, the initiator, the polymerization rate, and/or the intended application of the resultant polymer. In some embodiments, the reaction mixture is maintained at a specific temperature or within a range of temperatures to facilitate polymerization in the solution.

For example, and without being bound by theory, in some embodiments, when bulk polymerization is used, the temperature may be maintained from about 50° C. to about 85° C., including about 50° C., about 55° C., about 60° C., about 65° C., about 70° C., about 75° C., about 80° C., and about 85° C., including any range having any endpoint defined by any two of the aforementioned values. In some embodiments, the temperature may be maintained from about 60° C. to about 80° C.

In some embodiments, for example when solution polymerization is used, the temperature may be maintained from about 45° C. to about 70° C., including about 45° C., about 50° C., about 55° C., about 60° C., about 65° C., and about 70° C., including any range having any endpoint defined by any two of the aforementioned values. In some embodiments, the temperature may be maintained from about 50° C. to about 70° C.

In some embodiments, the polymerization method includes a chain termination step. In chain termination, the active growth of polymer chains is halted, leading to the formation of the high-strength copolymer. This phase of the polymerization process generally involve the cessation of chain elongation, resulting in the stabilization of the polymer chains at a particular length. It will be appreciated that the termination mechanism may vary depending on the type of polymerization process used.

For example, in radical polymerization, chain termination generally occurs through two primary mechanisms: combination and disproportionation. In the combination mechanism, two active polymer radicals react with each other to form a single, stable polymer chain. This reaction effectively ends the growth of both chains as the radical centers are neutralized, leading to the formation of a longer polymer chain without any active sites remaining. In the disproportionation mechanism, a radical chain ends by transferring a hydrogen atom from one polymer radical to another, resulting in the formation of two stable polymer chains with a terminal double bond and a saturated end group. Both mechanisms lead to the cessation of the polymerization process for the involved chains. In some embodiments, the termination mechanism is controlled through the polymerization conditions. For example, and without being bound by theory, in some embodiments, higher temperatures increase the rate of termination reactions, affecting the molecular weight and polymerization efficiency.

The efficiency and type of termination mechanism may influence the molecular weight, distribution, and overall properties of the copolymer. For example, a combination termination mechanism may produce a polymer with fewer terminal double bonds, whereas a disproportionation mechanism may result in polymers with more terminal functionalities, which may affect the polymer's properties and reactivity. It will be appreciated that controlling termination mechanisms and reaction conditions, such as temperature, monomer concentration, and the presence of impurities or inhibitors, allows for the tailoring of polymer properties for specific applications.

In some embodiments, the copolymer has a molecular weight of from about 20,000 Daltons to about 200,000 Daltons, including 25,000 Daltons, 30,000 Daltons, 40,000 Daltons, 50,000 Daltons, 60,000 Daltons, 70,000 Daltons, 80,000 Daltons, 90,000 Daltons, 100,000 Daltons, 110,000 Daltons, 120,000 Daltons, 125,000 Daltons, 130,000 Daltons, 140,000 Daltons, 150,000 Daltons, 160,000 Daltons, 170,000 Daltons, 180,000 Daltons, and 190,000 Daltons, including any range having endpoints by any two of the aforementioned values. In some embodiments, the molecular weight of the high-strength copolymer is 100,000 Daltons. It will be appreciated that the molecular weight of the copolymer is selected to ensure suitability of the copolymer for use in various manufacturing techniques. For example, the molecular weight may be selected to ensure flowability of the copolymer for molding, extrusion, and/or printing.

In some embodiments, the high-strength copolymer is recovered from solution. Any suitable means of recovery is contemplated and possible. For example, and not as a limitation, suitable methods of recovery include precipitation, solvent evaporation, steam stripping, anti-solvent addition, dialysis, centrifugation, and the like. In some embodiments, the acrylic carrier is precipitated out of the solution. In some embodiments, the copolymer is formed as granules or pellets.

In some embodiments, after recovery, the high-strength copolymer may undergo further purification to remove impurities, unreacted monomers, or additives. For example, in some embodiments, the high-strength copolymer may still contain residual solvent, monomer, long chain acrylic and/or other impurities, which may be removed by washing. Suitable washing solvents are solvents that do not dissolve the copolymer. In some embodiments, the high-strength copolymer is washed by pouring the washing solvent over it and allowing the washing solvent to percolate through a filter. In some embodiments, the high-strength copolymer undergoes multiple washing steps.

In some embodiments, the high-strength copolymer is dried to remove any residual solvent or moisture. Any suitable process for drying is contemplated and possible. Exemplary drying methods include, but are not limited to, air drying, vacuum drying, and thermal drying. It will be appreciated that the choice of drying method may depend on the copolymer's thermal stability and/or the boiling point of the residual solvent.

In some embodiment, further purification steps such as reprecipitation or dialysis may be necessary. Reprecipitation generally involves dissolving the polymer in a solvent and then reprecipitating by adding a non-solvent to remove impurities that were not eliminated in the initial precipitation. Dialysis may be employed for water-soluble polymers or when using aqueous systems to remove small-molecule impurities by diffusion through a semi-permeable membrane.

In some embodiments, the copolymer is combined with a plurality of ceramic particles to form a composite biomaterial. Optionally, the ceramic particles are biocompatible, bioactive materials used for repairing or replacing damaged bone. Optionally, the ceramic particles promote osteoconductivity and osteointegration. In embodiments, In some embodiments, the ceramic particles support new bone growth, and may interact with bone tissue when implanted to be totally integrated in several stages and eventually replaced by the newly formed bone. In some embodiments, the ceramic particles are a synthetic biomaterial that may include hydroxyapatite, tricalcium phosphate, bioglass, silicates, such as calcium silicate, sodium silicate, or silicate-substituted calcium phosphate, zirconia, combinations thereof, and the like.

In some embodiments, the ceramic particles are microparticles. Optionally, the ceramic particles range from about 50 μm to about 500 μm in diameter, including about 50 μm, about 60 μm, about 70 μm, about 80 μm, about 90 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm about 425 μm, about 450 μm, about 475 μm, about 500 μm, any range having endpoints defined by any two of the aforementioned values, and any subranges thereof. In some embodiments, the ceramic particles have a diameter of from about 100 μm to about 300 μm.

Optionally, the osteoactivity and/or osteoconductivity of the surgical implant is controlled by the amount of ceramic particles in the surgical implant. It will be further appreciated that adjusting the amount of ceramic particles may impacts the overall mechanical properties (e.g., structural integrity, load distribution, etc.) of the surgical implant, as well as the structural features (i.e., distribution of the ceramic particles, exposure of the ceramic granules through the polymer surface). In some embodiments, the ceramic particles are less than 50% of the total weight of the surgical implant. In some embodiments, the ceramic particles are less than 40% of the total weight of the surgical implant. In some embodiments, the ceramic particles are less than 30% of the total weight of the surgical implant.

Any suitable method for combining the ceramic particles with the copolymer is contemplated and possible. Suitable methods for mixing and processing the copolymer with ceramic particles include compounding extrusion, twin-screw extrusion, Banbury mixing, sigma mixers, roll mills, ribbon blenders, fluidized bed mixing, and high-shear mixers. In some embodiments, the copolymer is mixed with the ceramic particles and compounded.

In some embodiments, the copolymer is melted to form a molten copolymer. In some embodiments, the ceramic particles are added to the molten copolymer. In some embodiments, the molten copolymer and the ceramic particles are mixed to form a homogeneous mixture.

Optionally, the mixture is fed into a screw extruder, where it is heated and combined. In some embodiments, this mixing leads to a dispersion of the ceramic particles throughout the molten copolymer. In some embodiments, the ceramic particles are dispersed within the molten copolymer through mechanical shear forces generated by the screws. In some embodiments, the ceramic particles are uniformly dispersed within the copolymer, thereby enhancing mechanical stability and performance of the implant, described in greater detail herein.

In some embodiments, the composite biomaterial undergoes one or more of extrusion, pelletization, injection molding and/or additive manufacturing.

In some embodiments, the homogenized mixture exits the extruder through a die, forming a continuous filament, or other desired shape of extrusion (such as the desired implant shape, described in greater detail herein) to form an extrudate of the composite biomaterial. In some embodiments, the composite biomaterial is extruded to form a pellet, filament or bar. In some embodiments, the extrusion process involves pushing the mixture through a die to create uniform polymer-ceramic strands.

Optionally, the strands are cooled and pelletized. After extrusion, the extrudate may be cooled (e.g., by a water bath or air-cooling system) and solidified to stabilize its structure. Optionally, the extrudate is peeletized to form uniform cylindrical or spherical pellets. It will be appreciated that pelletization may be desirable in subsequent processing, such as molding, melting or compounding.

In some embodiments, the composite biomaterial is formed into the desired implant shape using extrusion techniques. Extrusion molding is a continuous manufacturing process, wherein the composite biomaterial is melted and forced through a die with a specific cross-sectional shape, for example a desired implant shape. The die design determines the initial geometry of the extrudate. In some embodiments, the extrudate may be further cut or machined into a final implant shape. The extrudate may undergo secondary processes such as precision machining, laser cutting, or surface texturing to create features like threads, holes, or porous surfaces needed for medical implants.

For example, and without being bound by theory, for dental implants, extrusion may be used to form base structures that may be later shaped into crowns, abutments, or anchors. Surface treatments like coating with bioactive substances may further enhance biocompatibility and promote osseointegration. Extrusion molding may be used to produce multiple implants with consistent quality and structural integrity, suitable for orthopedic rods, spinal cages, and/or dental frameworks. It will be appreciated that extrusion molding may be used in circumstances where a uniform implant shape across multiple implants is desired.

As described herein, the composite biomaterials of the present disclosure may be used to manufacture orthopedic and/or dental implants. In some embodiments, the surgical implant is customized to meet the specific needs of a subject. This customization may include tailoring the size, shape, surface characteristics, and/or materials to match the subject's anatomy and/or the clinical requirements of the procedure. For example, the implantation site may be imaged and measured, and the data used to guide the extrusion process through a die or to form a patient-specific mold, creating a surgical implant that is precisely sized and shaped to meet the subject's individual needs.

Customization of the surgical implant may be achieved through various techniques. For example, and without being bound by theory, in some embodiments, patient-specific imaging techniques such as CT scans, MRI, and 3D ultrasound may provide detailed anatomical data from the subject. This data may be used to generate 3D digital models for precise visualization and planning of the implant.

It will be further appreciated that patient-specific surgical planning may employ software for preoperative simulations, to create custom surgical guises, and position the osteoconductive composite implant accurately. In some embodiments, customization of the surgical implant may be adapted based on machine learning algorithms or other artificial intelligence models.

Optionally, the composite biomaterial is formed into the desired implant shape. In some embodiments, the composite biomaterial is formed into an implant using injection molding and/or additive manufacturing.

Optionally, the composite biomaterial is formed into an implant via injection molding. In some embodiments, the composite biomaterial is melted and injected into a mold cavity. Optionally, the injection happens at high pressure, thereby allowing the composite material to flow into all areas of the mold, capturing fine details and complex geometries. In some embodiments, the mold is designed such that the resulting implant will be configured to a subject's anatomical needs, described in greater detail herein. In some embodiments, for example when forming dental implants or other small orthopedic implants, microinjection molding may be used.

In some embodiments, the mold is maintained at a lower temperature to facilitate the cooling and solidification of the composite biomaterial once it is injected. In some embodiments, during the cooling phase, the material solidifies into the shape of the mold cavity, forming the implant. Post-molding modifications, such as surface treatments (e.g., plasma etching), coating with bioactive materials to improve osseointegration, and/or sterilization may be employed to enhance the implant's functionality.

Optionally, the composite biomaterial is formed into an implant via additive manufacturing. In some embodiments, the composite biomaterial is loaded into an additive manufacturing device, such as a 3D printer. Optionally, the composite biomaterial is heated to a molten or semi-solid state. In some embodiments, the heated composite biomaterial is extruded through a nozzle layer by layer to create the desired shape of the implant. It will be appreciated that the process allows for customized or patient-specific geometries that may be difficult to achieve with traditional molding methods.

In some embodiments, after polymerization and/or formation of the implant, the implant undergoes post-polymerization crosslinking to form the surgical implant. Post-polymerization crosslinking may be achieved through any suitable mechanism, such as heat, pressure, irradiation, or combinations thereof, though other mechanisms are contemplated and possible. Post-polymerization crosslinking generally induces permanent covalent bonds between polymer chains, forming a three-dimensional network structure. It will be appreciated that, in such embodiments, the formed implant is in a less crosslinked state than the final surgical implant. In some embodiments, the less crosslinked state allows melting, extrusion, and/or molding without degrading the polymer. In some embodiments, crosslinking the formed implant stabilizes the ceramic particles in the polymer matrix.

This post-polymerization step induces the formation of covalent bonds between the free functional groups within the polymer matrix, enhancing the implant's mechanical strength and fatigue resistance, rendering the implant suitable for load-bearing applications in orthopedics and serving to stabilize the ceramic particles. Optionally, post-polymerization crosslinking enhances the interfacial bonding and structural integrity between the ceramic particles and the polymer matrix. In some embodiments, the post-polymerization crosslinking enhances one or more mechanical, thermal, and/or chemical properties of the surgical implant relative to the formed implant, described in greater detail herein.

Optionally, the formed implant may be subjected to one or more processes which facilitate crosslinking, for example by generating or releasing free radicals, or by bringing the unreacted functional groups in closer proximity. Exemplary processes include heating, pressurization, and/or irradiation, described in greater detail herein. In some embodiments, the post-polymerization crosslinking is initiated by heat application to the formed implant. In some embodiments, the post-polymerization crosslinking is initiated by a combination of heat and/or pressure applied to the formed implant. In other embodiments the post-polymerization crosslinking is initiated by exposure to and/or free-radical generation via electromagnetic or mechanical irradiation, such as gamma irradiation, ultraviolet irradiation, microwave radiation, electron beam irradiation and/or infrared radiation.

In some embodiments, free radicals are generated by irradiation (e.g., UV light or gamma rays), heat, or chemical initiators. For example, and without being bound by theory, the free radicals may attack the vinyl groups (C═C) of the unreacted functional groups in the polymer chains, allowing the polymer chains to form covalent bonds and creating a network structure where the individual polymer chains crosslinked. Optionally, crosslinking may occur intermolecularly and/or intermolecularly.

In some embodiments, post-polymerization crosslinking of the implant involves diallyl crosslinking. For example, and without being bound by theory, in some embodiments, unreacted allyl groups on the bifunctional monomer and/or the long chain acrylic create covalent links between the polymer chains. In some embodiments, allyl groups from the allyl methacrylate and/or the polymethylmethacrylate react to crosslink the polymer chains. In some embodiments, allyl groups from the bifunctional monomer react to cross link the polymer chains. In some embodiments, allyl groups from the allyl methacrylate react to crosslink the polymer chains.

In some embodiments, a chemical catalyst is added to composite biomaterial to induce free radical formation within the formed implant. The catalyst may be introduced during the initial processing stages of the composite biomaterial, such as blending it with the molten copolymer when the ceramic particles are added. This catalyst may include peroxides, silanes, allyl compounds, UV initiators, and/or metal-based catalysts. It will be appreciated that organic peroxides may generate radicals upon heating, initiating the crosslinking reaction. Similarly, UV photoinitiators, activate under ultraviolet light to create free radicals that propagate crosslinking. If chemical crosslinking agents such as diallyl compounds are incorporated during the initial processing, they may be activated in subsequent post-processing steps to form covalent bonds between polymer chains.

Exemplary catalysts include, but are not limited to, peroxides (e.g., benzoyl peroxide, tert-butyl peroxide, dicumyl peroxide, etc.), azo compounds, (e.g., azobisisobutyronitrile, 2,2′-azobis (2-methylpropionamidine) dihydrochloride (V-50), 2,2′-Azobis [2-(2-imidazolin-2-yl) propane] dihydrochloride (VA-044), 2,2′-azobis (2-amidinopropane) dihydrochloride (V-511), etc.), transition metal catalysts, (e.g., cobalt naphthenate, platinum complexes, molybdenum catalysts, nickel catalysts, palladium catalysts, etc.), photoinitiators (e.g., benzoin, methyl ether, Igracure compounds, etc.), ionic catalysts (e.g., sulfuric acid, p-toluenesulfonic acid, sodium hydroxide, tertiary amines, etc.), diallyl crosslinkers (e.g., diallyl phthalate, diallyl isophthalate, diallyl carbonate, diallyl ether, etc.), combinations thereof, and the like, though any suitable catalyst is contemplated and possible.

In some embodiments, post-polymerization crosslinking of the implant is facilitated by heating. Optionally, the formed implant is placed in a controlled heating environment, such as an oven, press, or autoclave. In some embodiments, heating is performed in an inert atmosphere to prevent oxygen degradation of the polymer. Optionally, heat is applied to the formed implant at a predetermined temperature for a given time period. In some embodiments, the predetermined temperature is from about 100° C. to about 300° C., including about 100° C., about 110° C., about 120° C., about 125° C., about 130° C., about 140° C., about 150° C., about 160° C., about 170° C., about 175° C., about 180° C., about 190° C., about 200° C., about 210° C., about 220° C., about 225° C., about 230° C., about 240° C., about 250° C., about 260° C., about 270° C., about 275° C., about 280° C., about 290° C., or about 300° C., including any range having endpoints by any two of the aforementioned values. In some embodiments, the temperature is maintained for a time period of from about 30 minutes to about 5 hours, including about 30 minutes, about 45 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, or about 5 hours, including any range having endpoints by any two of the aforementioned values. In some embodiments, the formed implant is heated simultaneously with pressure application. In some embodiments, the formed implant is heated and irradiated simultaneously. In some embodiments, the formed implant is heated, irradiated, and pressurized simultaneously.

In some embodiments, post-polymerization crosslinking of the implant is facilitated by pressure. Optionally, the formed implant is placed in a high-pressure chamber capable of maintaining uniform pressure and temperature. Optionally, pressure is applied to the formed implant at a predetermined pressure for a given time period. In some embodiments, the predetermined pressure is from about 50 MPa to about 250 MPa, including about 50 MPa, about 60 MPa, about 70 MPa, about 80 MPa, about 90 MPa, about 100 MPa, about 110 MPa, about 120 MPa, about 125 MPa, about 130 MPa, about 140 MPa, about 150 MPa, about 160 MPa, about 170 MPa, about 180 MPa, about 190 MPa, about 200 MPa, about 210 MPa, about 220 MPa, about 225 MPa, about 230 MPa, about 240 MPa, or about 250 MPa, including any range having endpoints by any two of the aforementioned values. In some embodiments, the pressure is maintained for a time period of from about 30 minutes to about 5 hours, including about 30 minutes, about 45 minutes, about 1 hour, about 1.25 hours, about 1.5 hours, about 1.75 hours, about 2 hours, about 2.25 hours, about 2.5 hours, about 2.75 hours, about 3 hours, about 3.25 hours, about 3.5 hours, about 3.75 hours, about 4 hours, about 4.25 hours, about 4.5 hours, about 4.75 hours, or about 5 hours, including any range having endpoints by any two of the aforementioned values. In some embodiments, the formed implant is heated simultaneously with pressure application.

In some embodiments, post-polymerization crosslinking of the implant is facilitated by irradiation. Optionally, the post-polymerization crosslinking also sterilizes the surgical implant. Any suitable irradiation, such as gamma irradiation, ultraviolet irradiation, microwave irradiation, electron beam irradiation, infrared irradiation, combinations thereof, and the like, are contemplated and possible. It will be appreciated that irradiation generates free radicals, which thereby facilitate post-polymerization crosslinking.

In some embodiments, post-polymerization crosslinking of the implant is facilitated by gamma irradiation. Optionally, high-energy gamma rays ionize atoms and molecules in the polymer, generating free radicals in the polymer chains. In some embodiments, the formed implant is irradiated at an energy level of from about 0.1 MeV to about 10 MeV, including about 0.5 MeV, about 1.0 MeV, about 1.5 MeV, about 2.0 MeV, about 2.5 MeV, about 3 MeV, about 3.5 MeV, about 4.0 MeV, about 4.5 MeV, about 5 MeV, about 5.5 MeV, about 6.0 MeV, about 6.5 MeV, about 7.0 MeV, about 7.5 MeV, about 8.0 MeV, about 8.5 MeV, about 9.0 MeV, about 9.5 MeV, or about 10.0 MeV, including any range having endpoints by any two of the aforementioned values. In some embodiments, the formed implant is irradiated with gamma radiation simultaneously with pressure application. In some embodiments, the formed implant is irradiated with gamma radiation simultaneously with heating. In some embodiments, the formed implant is irradiated with gamma radiation simultaneously with pressure application and heating.

In some embodiments, post-polymerization crosslinking of the implant is facilitated by irradiation with ultraviolet (UV) light. Optionally, low-energy UV waves excite double bonds in the unreacted functional groups, generating free radicals in the polymer chains. In some embodiments, the formed implant is irradiated with UV waves having a wavelength from about 200 nm to about 400 nm, including about 200 nm, about 225 nm, about 250 nm, about 275 nm, about 300 nm, about 325 nm, about 350 nm, about 375 nm, or about 400 nm, including any range having endpoints by any two of the aforementioned values. In some embodiments, the formed implant is irradiated with UV radiation simultaneously with pressure application. In some embodiments, the formed implant is irradiated with UV radiation simultaneously with heating. In some embodiments, the formed implant is irradiated with UV radiation simultaneously with pressure application and heating.

In some embodiments, post-polymerization crosslinking of the implant is facilitated by microwave irradiation. Optionally, microwaves heat the polymer matrix, generating free radicals. In some embodiments, the formed implant is irradiated with microwaves having a frequency of about 2.45 GHz. In some embodiments, the formed implant is irradiated with microwaves simultaneously with pressure application. In some embodiments, the formed implant is irradiated with microwaves simultaneously with heating. In some embodiments, the formed implant is irradiated with microwaves simultaneously with pressure application and heating.

In some embodiments, post-polymerization crosslinking of the implant is facilitated by electron beam irradiation. Optionally, high-energy electrons are accelerated and directed at the formed implant. These electrons ionize the polymer matrix, generating free radicals. In some embodiments, the formed implant is irradiated with an electron beam at an energy level of from about 0.1 MeV to about 10 MeV, including about 0.5 MeV, about 1.0 MeV, about 1.5 MeV, about 2.0 MeV, about 2.5 MeV, about 3 MeV, about 3.5 MeV, about 4.0 MeV, about 4.5 MeV, about 5 MeV, about 5.5 MeV, about 6.0 MeV, about 6.5 MeV, about 7.0 MeV, about 7.5 MeV, about 8.0 MeV, about 8.5 MeV, about 9.0 MeV, about 9.5 MeV, or about 10.0 MeV, including any range having endpoints by any two of the aforementioned values. In some embodiments, the formed implant is irradiated with electron beam radiation simultaneously with pressure application. In some embodiments, the formed implant is irradiated with electron beam radiation simultaneously with heating. In some embodiments, the formed implant is irradiated with electron beam radiation simultaneously with pressure application and heating.

In some embodiments, post-polymerization crosslinking of the implant is facilitated by irradiation with infrared light. Optionally, infrared waves heat the formed implant, generating free radicals in the polymer chains. In some embodiments, the formed implant is irradiated with infrared waves having a wavelength from 0.75 μm to about 1000 μm, including about 0.75 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, about 700 μm, about 725 μm, about 750 μm, about 775 μm, about 800 μm, about 825 μm, about 850 μm, about 875 μm, about 900 μm, about 925 μm, about 950 μm, about 975 μm, or about 1000 μm, including any range having endpoints by any two of the aforementioned values. In some embodiments, the formed implant is irradiated with UV radiation simultaneously with pressure application. In some embodiments, the formed implant is irradiated with UV radiation simultaneously with heating. In some embodiments, the formed implant is irradiated with UV radiation simultaneously with pressure application and heating.

In some embodiments, the degree of crosslinking may be modulated. It will be appreciated that temperature and/or duration may be adapted to modulate the degree of crosslinking. For example, and without being bound by theory, in some embodiments, higher temperatures may increase the mobility of polymer chains and accelerate the reaction, leading to more crosslinking. Conversely, in some embodiments, lower temperatures may slow the reaction and result in fewer crosslinks. Similarly, in some embodiments, extending the heating duration may allow more time for crosslinking reactions to occur, while shorter durations may limit the extent of crosslinking.

Optionally, the surrounding atmosphere may be adapted to modulate the degree of crosslinking. For example, and without being bound by theory, in some embodiments, conducting post-polymerization crosslinking in the presence of an inert gas (i.e., nitrogen) may prevent oxygen from quenching free radicals, thereby enhancing crosslinking, whereas allowing some oxygen exposure may naturally limit the reaction.

In some embodiments, irradiation conditions may also be adjusted to modulate crosslinking. For example, and without being bound by theory, different types of irradiation, such as gamma rays, electron beams, UV, or infrared light, may offer varying levels of penetration and effect. In some embodiments, gamma and/or electron beams penetrate the formed implant deeply and create uniform crosslinking, while UV and/or infrared are more surface-focused, allowing for localized control of crosslinking. The energy level or dosage of irradiation may impact crosslink density. For example, higher energy levels may generate more radicals, resulting in higher crosslinking, while lower energy levels may produce moderate crosslinking. Further, as described herein, in some embodiments, varying exposure time may modulate the degree of crosslinking, for example, with longer durations enhancing crosslinking and shorter durations limiting it.

In some embodiments, post-polymerization crosslinking of the formed implant results in a surgical implant with enhanced mechanical, thermal, or chemical properties relative to the pre-crosslinked state of the formed implant. For example, and without being bound by theory, in some embodiments, the formation of a cross-linked network restricts polymer chain mobility, which prevents excessive elongation or compression under stress. In some embodiments, the network may also aid in distributing applied forces evenly throughout the structure, reducing the likelihood of failure at weak points. Optionally, crosslinking may enhance chain integrity by preventing molecular slippage, thereby maintaining structural stability under stress.

Optionally, crosslinking the implant enhances the strength, fatigue resistance, durability, and/or stability of the implant for use in load-bearing applications. It will be appreciated that the properties of the surgical implant, such as tensile strength and/or compressive strength, necessary for load-bearing applications in orthopedics and/or dentistry may vary depending on the specific applications, the materials being used, and the degree of crosslinking.

In some embodiments, load-bearing orthopedic implants, may be designed to endure significant mechanical stresses during activities like walking, running, and/or lifting. For example, and without being bound by theory, hip and knee joint replacements must withstand compressive forces exceeding 2,000 N during activities like walking. Bone plates and screws require high tensile strength to resist bending or breaking under dynamic load. Similarly, dental implants and restorative materials, such as crowns and bridges, are subject to the forces of biting and chewing, which in humans can range from 200 to 500 N. In some embodiments, dental implants possess both high tensile and compressive strength to ensure stable anchorage in bone and effective load transfer during mastication and to resist occlusal forces without cracking or deforming, ensuring durability and functionality in the oral environment.

In some embodiments, the mechanical, thermal, and/or chemical properties of the implant match or exceed the associated properties of the surrounding bone to prevent stress shielding and/or implant failure. Optionally, surgical implants according to the present disclosure may withstand 3 to 10 times the expected physiological loads, to withstand high-impact activities without failing.

In some embodiments, post-polymerization crosslinking enhances one or more mechanical properties of the surgical implant relative to the formed implant. For example, and without being bound by theory, cross-linking may enhance one or more mechanical properties of polymers by introducing covalent bonds that restrict chain mobility. In some embodiments, post-polymerization crosslinking results in improved tensile strength and stiffness, allowing the surgical implant to resist higher loads without deforming. In some embodiments, the surgical implant exhibits enhanced elasticity and creep resistance, demonstrating elastic recovery and maintaining structural integrity under sustained loads, even in the presence of heat or pressure. Impact resistance and abrasion resistance may also be improved, enabling the surgical implant to endure sudden mechanical forces and wear, making it well-suited for high-stress applications. Exemplary, non-limiting mechanical properties that may be enhanced include tensile strength, compressive strength, elastic modulus, toughness, impact resistance, tear strength, abrasion resistance, hardness, creep resistance, fatigue resistances, and/or dimensional stability.

In some embodiments, the surgical implant demonstrates increased tensile strength relative to the formed implant. Tensile strength, the maximum stress a material can endure while being stretched or pulled before breaking, may be enhanced through post-polymerization cross-linking. For example, and without being bound by theory, post-polymerization crosslinking may reinforce the molecular structure, increasing the polymer's ability to withstand tensile forces. In some embodiments, post-polymerization crosslinking enhances the tensile strength of the surgical implant relative to the formed implant from about 20% to about 100%, including about 20%, about 25%, about 30%, about 40%, about 45%, about 50%, about 55%, about 60%, about 65%, about 70%, about 75%, about 80%, about 85%, about 90%, about 95%, or about 100%, including any range having endpoints defined by any two of the aforementioned values. In some embodiments, moderate levels of post-polymerization crosslinking improve the tensile strength of the surgical implant by about 20% to about 50% relative to the formed implant. In some embodiments, high levels of post-polymerization crosslinking improve the tensile strength of the surgical implant by about 60% to about 100% relative to the formed implant.

In some embodiments, the surgical implant has a tensile strength from about 50 MPa to about 1500 MPa, including about 50 MPa, about 75 MPa, about 100 MPa, about 125 MPa, about 150 MPa, about 175 MPa, about 200 MPa, about 225 MPa, about 250 MPa, about 275 MPa, about 300 MPa, about 325 MPa, about 350 MPa, about 375 MPa, about 400 MPa, about 425 MPa, about 450 MPa, about 475 MPa, about 500 MPa, about 525 MPa, about 550 MPa, about 575 MPa, about 600 MPa, 625 MPa, about 650 MPa, about 675 MPa, about 700 MPa, about 725 MPa, about 750 MPa, about 800 MPa, about 825 MPa, about 850 MPa, about 875 MPa, about 900 MPa, about 975 MPa, about 1000 MPa, about 1050 MPa, about 1100 MPa, about 1150 MPa, about 1200 MPa, about 1250 MPa, about 1300 MPa, about 1350 MPa, about 1400 MPa, about 1450 MPa, or about 1500 MPa, including any range having endpoints defined by any two of the aforementioned values.

In some embodiments, the surgical implant is an orthopedic implant, having a tensile strength of from about 100 MPa to about 800 MPa, including about 100 MPa, about 125 MPa, about 150 MPa, about 175 MPa, about 200 MPa, about 225 MPa, about 250 MPa, about 275 MPa, about 300 MPa, about 325 MPa, about 350 MPa, about 375 MPa, about 400 MPa, about 425 MPa, about 450 MPa, about 475 MPa, about 500 MPa, about 525 MPa, about 550 MPa, about 575 MPa, about 600 MPa, 625 MPa, about 650 MPa, about 675 MPa, about 700 MPa, about 725 MPa, about 750 MPa, about 775 MPa or about 800 MPa, including any range having endpoints defined by any two of the aforementioned values.

In some embodiments, the surgical implant is a dental implant, having a tensile strength of from about 50 MPa to about 300 MPa, including about 50 MPa, about 75 MPa, about 100 MPa, about 125 MPa, about 150 MPa, about 175 MPa, about 200 MPa, about 225 MPa, about 250 MPa, about 275 MPa, or about 300 MPa, including any range having endpoints defined by any two of the aforementioned values.

In some embodiments, the surgical implant demonstrates increased compressive strength relative to the formed implant. Compressive strength refers to a material's ability to resist deformation under compressive forces, which is enhanced by post-polymerization crosslinking. For example, and without being bound by theory, post-polymerization crosslinking may aid in creating a rigid framework that supports higher loads without collapsing. In some embodiments, post-polymerization crosslinking enhances the compressive strength of the surgical implant relative to the formed implant from about 50% to about 200%, including about, about 50%, about 55%, about 60%, about 65%, about 70%, about 75%, about 80%, about 85%, about 90%, about 95%, about 100%, about 125%, about 150%, about 175%, or about 200%, including any range having endpoints defined by any two of the aforementioned values.

In some embodiments, the surgical implant has a compressive strength from about 100 MPa to about 3000 MPa, including about 100 MPa, about 125 MPa, about 150 MPa, about 175 MPa, about 200 MPa, about 225 MPa, about 250 MPa, about 275 MPa, about 300 MPa, about 325 MPa, about 350 MPa, about 375 MPa, about 400 MPa, about 425 MPa, about 450 MPa, about 475 MPa, about 500 MPa, about 525 MPa, about 550 MPa, about 575 MPa, about 600 MPa, 625 MPa, about 650 MPa, about 675 MPa, about 700 MPa, about 725 MPa, about 750 MPa, about 800 MPa, about 825 MPa, about 850 MPa, about 875 MPa, about 900 MPa, about 975 MPa, about 1000 MPa, about 1050 MPa, about 1100 MPa, about 1150 MPa, about 1200 MPa, about 1250 MPa, about 1300 MPa, about 1350 MPa, about 1400 MPa, about 1450 MPa, about 1500 MPa, about 1550 MPa, about 1600 MPa, about 1650 MPa, about 1700 MPa, about 1750 MPa, about 1800 MPa, about 1850 MPa, about 1900 MPa, about 2000 MPa, about 2100 MPa, about 2200 MPa, about 2250 MPa, about 2300 MPa, about 2400 MPa, about 2500 MPa, about 2600 MPa, about 2700 MPa, about 2750 MPa, about 2800 MPa, about 2900 MPa, or about 3000 MPa including any range having endpoints defined by any two of the aforementioned values

In some embodiments, the surgical implant is an orthopedic implant, having a compressive strength of from about 100 MPa to about 1000 MPa, including about 100 MPa, about 125 MPa, about 150 MPa, about 175 MPa, about 200 MPa, about 225 MPa, about 250 MPa, about 275 MPa, about 300 MPa, about 325 MPa, about 350 MPa, about 375 MPa, about 400 MPa, about 425 MPa, about 450 MPa, about 475 MPa, or about 500 MPa, about 525 MPa, about 550 MPa, about 575 MPa, about 600 MPa, 625 MPa, about 650 MPa, about 675 MPa, about 700 MPa, about 725 MPa, about 750 MPa, about 775 MPa, about 800 MPa, about 825 MPa, about 850 MPa, about 875 MPa, about 900 MPa, about 975 MPa, or about 1000 MPa, including any range having endpoints defined by any two of the aforementioned values.

In some embodiments, the surgical implant is a dental implant, having a tensile strength of from about 150 MPa to about 2500 MPa, including about 150 MPa, about 175 MPa, about 200 MPa, about 225 MPa, about 250 MPa, about 275 MPa, about 300 MPa, about 325 MPa, about 350 MPa, about 375 MPa, about 400 MPa, about 425 MPa, about 450 MPa, about 475 MPa, about 500 MPa, about 525 MPa, about 550 MPa, about 575 MPa, about 600 MPa, 625 MPa, about 650 MPa, about 675 MPa, about 700 MPa, about 725 MPa, about 750 MPa, about 800 MPa, about 825 MPa, about 850 MPa, about 875 MPa, about 900 MPa, about 975 MPa, about 1000 MPa, about 1050 MPa, about 1100 MPa, about 1150 MPa, about 1200 MPa, about 1250 MPa, about 1300 MPa, about 1350 MPa, about 1400 MPa, about 1450 MPa, about 1500 MPa, about 1550 MPa, about 1600 MPa, about 1650 MPa, about 1700 MPa, about 1750 MPa, about 1800 MPa, about 1850 MPa, about 1900 MPa, about 2000 MPa, about 2100 MPa, about 2200 MPa, about 2250 MPa, about 2300 MPa, about 2400 MPa, or about 2500 MPa, including any range having endpoints defined by any two of the aforementioned values.

In some embodiments, the surgical implant demonstrates improved elastic modulus relative to the formed implant. Optionally, post-polymerization crosslinking restricts molecular mobility. In some embodiments, the elastic modulus of the surgical implant is improved from 30% to 150% relative to the formed implant. In some embodiments, the surgical implant demonstrates improved toughness and/or impact resistance relative to the formed implant. Post-polymerization crosslinking may allow the surgical implant to absorb more energy before fracturing. In some embodiments, toughness is increased from about 20% to about 60% relative to the formed implant.

In some embodiments, the surgical implant may demonstrate enhanced creep resistance, reducing slow, permanent deformation of the implant under sustained loads. The surgical implant may demonstrate improved fatigue resistance, enabling the surgical implant to endure repeated mechanical stresses without failure. Dimensional stability may also be improved, with the fixed cross-linked structure maintaining integrity under mechanical and thermal stresses.

Post-polymerization crosslinking may enhance one or more thermal properties of the surgical implant relative to the pre-crosslinked formed implant. In some embodiments, post-polymerization crosslinking stabilizes the polymer matrix, thereby making the surgical implant more resistant to thermal degradation. In some embodiments, the surgical implant demonstrates improved thermal stability. Optionally, post-polymerization crosslinking of the surgical implant increases the decomposition temperature of the polymer by about 50° C. to about 150° C. In some embodiments, post-polymerization crosslinking increases the glass transition temperature. Optionally, the surgical implant may demonstrate a glass transition temperature from about 10° C. to about 50° C. higher than the formed implant, enabling the surgical implant to maintain its properties at higher temperatures. Crosslinking may also enhances the heat distortion temperature (HDT). Optionally, the surgical implant has an HDT of about 20° C. to about 100° C. higher than the formed implant. In some embodiments, post-polymerization cross-linking prevents or reduces flow or deformation under continuous thermal stress, thereby increasing resistance to thermal creep. In some embodiments, post-polymerization crosslinking reduces flammability by creating a more thermally stable, char-forming structure, thereby improving flame retardance. Additional thermal properties that may be enhanced include, but are not limited to melting temperature, thermal conductivity, reduction of the coefficient of thermal expansion (CTE), leading to lower dimensional changes with temperature fluctuations, reduction of thermal shrinkages, and the like.

In some embodiments, post-polymerization crosslinking may enhance one or more chemical properties of the surgical implant, for example by improving its resistance to solvents, oxidative degradation, and/or moisture absorption. In some embodiments, the surgical implant demonstrates a 50% to 300% improvement in chemical resistance relative to the formed implant, allowing the surgical implant to withstand a wider range of chemicals such as enzymes, acids, bases, oils, and the like.

In some embodiments, the surgical implant may exhibit enhanced swelling resistance and/or improved barrier properties, such as permeability. Optionally, post-polymerization crosslinking restricts the penetration of liquids, minimizing swelling in solvent-rich environments. In some embodiments, the surgical implant exhibits decreased swelling from about 30% to about 200%. In some embodiments, the surgical implants demonstrate reduced solvent uptake from about 30% to about 70%. The surgical implant may also show increased resistance to oxidation and/or hydrolysis.

The surgical implants of the present disclosure demonstrate enhanced mechanical, thermal, and/or chemical properties relative to their unlinked counterparts. Post-polymerization crosslinking improves tensile strength, compressive strength, and elastic modulus by restricting chain mobility and enhancing force distribution. Post-polymerization crosslinking also raises the glass transition temperature, HDT, and thermal stability, resulting in a surgical implant that is more heat-resistant and less prone to deformation or degradation under elevated temperatures. Additionally, the surgical implants demonstrate improved solvent resistance, oxidative stability, chemical resistance, and reduced water absorption, making them more durable when implanted in a subject.

### Aspects Listing

In a first aspect, alone or in combination with any other aspect herein, the present disclosure relates to a method of manufacturing a surgical implant, the method comprising polymerizing a bifunctional monomer with a long-chain acrylic to form a high-strength copolymer; dispersing a plurality of ceramic particles in the copolymer to form a composite biomaterial; forming the composite biomaterial into an implant; and crosslinking the formed implant to form the surgical implant, thereby stabilizing the ceramic particles in the surgical implant.

In a second aspect, alone or in combination with any other aspect herein, the present disclosure relates to a method of manufacturing a surgical implant, wherein the surgical implant is an orthopedic implant or dental implant.

In a third aspect, alone or in combination with any other aspect herein, the present disclosure relates to a method of manufacturing a surgical implant, wherein the bifunctional monomer is selected from allyl methacrylate, vinyl acetate, ethylene glycol dimethacrylate, 1,4-butanediol dimethacrylate, glycidal methacrylate, divinylbenzene, and combinations thereof.

In a fourth aspect, alone or in combination with any other aspect herein, the present disclosure relates to a method of manufacturing a surgical implant, wherein the bifunctional monomer is allyl methacrylate.

In a fifth aspect, alone or in combination with any other aspect herein, the present disclosure relates to a method of manufacturing a surgical implant, wherein the long-chain acrylic is selected from polymethyl methacrylate, polymethyl methacrylate, polybutyl methacrylate, polylaurel methacrylate, polyethylene methacrylate, polystyrene methacrylate, polymethyl acrylate, and combinations thereof.

In a sixth aspect, alone or in combination with any other aspect herein, the present disclosure relates to a method of manufacturing a surgical implant, wherein the long-chain acrylic is polymethylmethacrylate.

In a seventh aspect, alone or in combination with any other aspect herein, the present disclosure relates to a method of manufacturing a surgical implant, wherein the high-strength polymer has a molecular weight of about 100,000 Daltons.

In an eighth aspect, alone or in combination with any other aspect herein, the present disclosure relates to a method of manufacturing a surgical implant, wherein the ceramic particles are selected from hydroxyapatite, tricalcium phosphate, bioglass, silicates and zirconia.

In a ninth aspect, alone or in combination with any other aspect herein, the present disclosure relates to a method of manufacturing a surgical implant, wherein crosslinking the formed implant comprises diallyl crosslinking.

In a tenth aspect, alone or in combination with any other aspect herein, the present disclosure relates to a method of manufacturing a surgical implant, wherein crosslinking is initiated by applying heat, pressure, irradiation, or a combination thereof to the formed implant.

In an eleventh aspect, alone or in combination with any other aspect herein, the present disclosure relates to a method of manufacturing a surgical implant, wherein the irradiation is selected from gamma irradiation, ultraviolet irradiation, microwave radiation, electron beam irradiation, infrared radiation, and combinations thereof.

In a twelfth aspect, alone or in combination with any other aspect herein, the present disclosure relates to a method of manufacturing a surgical implant, wherein the high-strength copolymer is polymerized with a catalyst to facilitate crosslinking.

In a thirteenth aspect, alone or in combination with any other aspect herein, the present disclosure relates to a method of manufacturing a surgical implant, wherein the catalyst is selected from peroxides, UV initiators, or metal-based catalysts.

In a fourteenth aspect, alone or in combination with any other aspect herein, the present disclosure relates to a method of manufacturing a surgical implant, wherein crosslinking the formed implant enhances one or more properties of the surgical implant relative to the formed implant.

In a fifteenth aspect, alone or in combination with any other aspect herein, the present disclosure relates to a method of manufacturing a surgical implant, wherein the one or more properties are selected from mechanical properties, thermal properties, or chemical properties.

In a sixteenth aspect, alone or in combination with any other aspect herein, the present disclosure relates to a method of manufacturing a surgical implant, wherein the mechanical properties are selected from increased tensile strength or increased compressive strength.

In a seventeenth aspect, alone or in combination with any other aspect herein, the present disclosure relates to a method of manufacturing a surgical implant, wherein the plurality of ceramic particles are dispersed in the copolymer when the copolymer is in a molten state.

In an eighteenth aspect, alone or in combination with any other aspect herein, the present disclosure relates to a method of manufacturing a surgical implant, wherein forming the implant comprises injection molding, extrusion, pelletization, or additive manufacturing.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” and “about” are used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

Unless otherwise defined, all technical and scientific terms used herein have the same meaning as commonly understood by one of ordinary skill in the art to which the invention belongs. The terminology used in the description herein is for describing particular embodiments only and is not intended to be limiting. As used in the specification and appended claims, the singular forms “a,” “an,” and “the” are intended to include the plural forms as well, unless the context clearly indicates otherwise.

As used herein, the terms “horizontal” and “vertical” are relative terms only, are indicative of a general relative orientation only, and do not necessarily indicate perpendicularity. These terms also may be used for convenience to refer to orientations used in the figures, which orientations are used as a matter of convention only and are not intended as characteristic of the devices shown. The present invention and the embodiments thereof to be described herein may be used in any desired orientation. Moreover, horizontal and vertical walls need generally only be intersecting walls, and need not be perpendicular.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method of manufacturing a surgical implant, the method comprising:
polymerizing a bifunctional monomer with a long-chain acrylic to form a high-strength copolymer;
dispersing a plurality of ceramic particles in the copolymer to form a composite biomaterial;
forming the composite biomaterial into an implant; and
crosslinking the formed implant to form the surgical implant, thereby stabilizing the ceramic particles in the surgical implant.

2. The method of claim 1, wherein the surgical implant is an orthopedic implant or dental implant.

3. The method of claim 1, wherein the bifunctional monomer is selected from allyl methacrylate, vinyl acetate, ethylene glycol dimethacrylate, 1,4-butanediol dimethacrylate, glycidal methacrylate, divinylbenzene, and combinations thereof.

4. The method of claim 1, wherein the bifunctional monomer is allyl methacrylate.

5. The method of claim 1, wherein the long-chain acrylic is selected from polymethyl methacrylate, polymethyl methacrylate, polybutyl methacrylate, polylaurel methacrylate, polyethylene methacrylate, polystyrene methacrylate, polymethyl acrylate, and combinations thereof.

6. The method of claim 1, wherein the long-chain acrylic is polymethyl methacrylate.

7. The method of claim 1, wherein the high-strength polymer has a molecular weight of about 100,000 Daltons.

8. The method of claim 1, wherein the ceramic particles are selected from hydroxyapatite, tricalcium phosphate, bioglass, silicates and zirconia.

9. The method of claim 1, wherein crosslinking the formed implant comprises diallyl crosslinking.

10. The method of claim 1, wherein crosslinking is initiated by applying heat, pressure, irradiation, or a combination thereof to the formed implant.

11. The method of claim 10, wherein the irradiation is selected from gamma irradiation, ultraviolet irradiation, microwave radiation, electron beam irradiation, infrared radiation, or combinations thereof.

12. The method of claim 1, wherein the composite biomaterial comprises a catalyst to facilitate crosslinking.

13. The method of claim 12, wherein the catalyst is selected from peroxides, UV initiators, or metal-based catalysts.

14. The method of claim 1, wherein crosslinking the formed implant enhances one or more properties of the surgical implant relative to the formed implant.

15. The method of claim 14, wherein the one or more properties are selected from mechanical properties, thermal properties, or chemical properties.

16. The method of claim 15, wherein the mechanical properties are selected from increased tensile strength or increased compressive strength.

17. The method of claim 1, wherein the plurality of ceramic particles are dispersed in the copolymer when the copolymer is in a molten state.

18. The method of claim 1, wherein forming the implant comprises injection molding, extrusion, pelletization, or additive manufacturing.
